(19) United States
(12) Patent Application Publication
(10) Pub. No.: US 2022/0087613 A1
(43) Pub. Date: Mar. 24, 2022

(54) HEALTH SENSING BATHROOM DEVICE

(71) Applicant: Kohler Co., Kohler, WI (US)

(72) Inventors: Rafael Rexach, Sheboygan, WI (US); Jiunntyng Chen, Kohler, WI (US)

(21) Appl. No.: 17/468,266

(22) Filed: Sep. 7, 2021

Related U.S. Application Data
(60) Provisional application No. 63/082,237, filed on Sep. 23, 2020.
(60) Provisional application No. 63/128,679, filed on Dec. 21, 2020.

(51) Int. Cl. A61B 5/00; A61B 5/0205; A61B 5/024; A61B 5/08; A61B 5/107; A61B 5/1171

(52) U.S. Cl. CPC A61B 5/6891; A61B 5/0046; A61B 5/0205; A61B 5/02405; A61B 2560/04; A61B 5/1073; A61B 5/1176; A61B 5/742; A61B 5/0816

(57) ABSTRACT

Various bathroom devices for the collection of data are described. In one example, a mirror cabinet includes a mirror frame, a sensor cavity, and a controller. The mirror frame is configured to support a mirror substrate that provides a reflection of one or more users in proximity of the mirror cabinet. The sensor cavity coupled to the mirror frame and is configured to support a sensor for detecting a health condition of the one or more users in proximity to the mirror cabinet. A controller is configured to analyze data received from the sensor to determine the health condition.

### CROSS-REFERENCE TO RELATED APPLICATIONS

This application claims priority benefit to U.S. Provisional Utility Application Ser. No. 63/082,237 (Docket No. 010222-20032A) filed on Sep. 23, 2020, and U.S. Provisional Utility Application Ser. No. 63/128,679 (Docket No. 010222-20032B) filed on Dec. 21, 2020. The entire disclosure of each is hereby incorporated by reference.

### FIELD

The present application relates generally to bathroom devices configured for the collection of data.

### BACKGROUND

The collection of physical data on a human may present several challenges. One example is maintaining a consistent source of data. For example, the availability of users at the same time and place every day is not reliable. Few people, for example, take their temperature or blood pressure on a daily basis. Thus, even if a thermometer or a blood pressure cuff includes communication devices to remote report data, achieving consistent use of the thermometers or blood pressure cuffs may be a challenge.

### BRIEF DESCRIPTION OF THE DRAWINGS

Exemplary embodiments are described herein with reference to the following drawings, according to an exemplary embodiment.

FIG. 1 illustrates an example health sensing mirror and computer network.

FIG. 2 illustrates another example health sensing mirror.

FIG. 3 illustrates a proximity trigger for a retractable sensor assembly of an example health sensing mirror.

FIG. 4 illustrates a field of view for a camera of the example health sensing mirror.

FIG. 5 illustrates an opened retractable sensor assembly.

FIG. 6 illustrates a closed retractable sensor assembly.

FIG. 7 illustrates an example sanitizer dispenser.

FIG. 8 illustrates a cartridge for the sanitizer dispenser.

FIGS. 9-11 illustrate example modules for a mirror.

FIG. 12 illustrates an example toilet with a health sensing toilet seat.

FIG. 13 illustrates the health sensing toilet seat.

FIG. 14 illustrates an example display for a health sensing toilet seat.

FIG. 15 illustrates example images for the display for the health sensing toilet seat.

FIG. 16 illustrates an example embodiment for the controller for the health sensing mirror or toilet seat.

FIG. 17 illustrates an example mobile device interface for the health sensing mirror or health sensing toilet seat.

FIG. 18 illustrates an example mobile device interface for the health sensing mirror or health sensing toilet seat.

FIG. 19 illustrates a flow chart for the controller of FIG. 16.

FIG. 20 illustrates another flow chart for the controller of FIG. 16.

### DETAILED DESCRIPTION

The following embodiments include one or more devices, traditionally located in the bathroom, that include one or more sensors configured for the collection of data related to the physical state of the user. The devices may include cabinets for use in bathrooms and the like (e.g., medicine cabinets or mirror cabinets) and to toilet seats or other surfaces that contact the user in the bathroom, although the concepts disclosed herein may also be used in other locations and for other purposes.

For many people, their daily routine includes at least one brief stop in front of a mirror, such as a bathroom mirror or an entry way mirror. The mirror may be mounted to a wall and extend outward from the wall. The mirror be mounted within the wall (e.g., between studs) and be flush, or nearly flush, with the surface of the wall. The mirror may be harnessed as a portal into the lives of users. The users may visit the mirror to view their reflection or perform other personal hygiene functions. These visits provide an opportunity to collect data from the users on a regular basis. The data may describe the health of the user. Tracking the health of the user over time provides a wealth of opportunities for technological improvements in a variety of technology areas as described in the following embodiments.

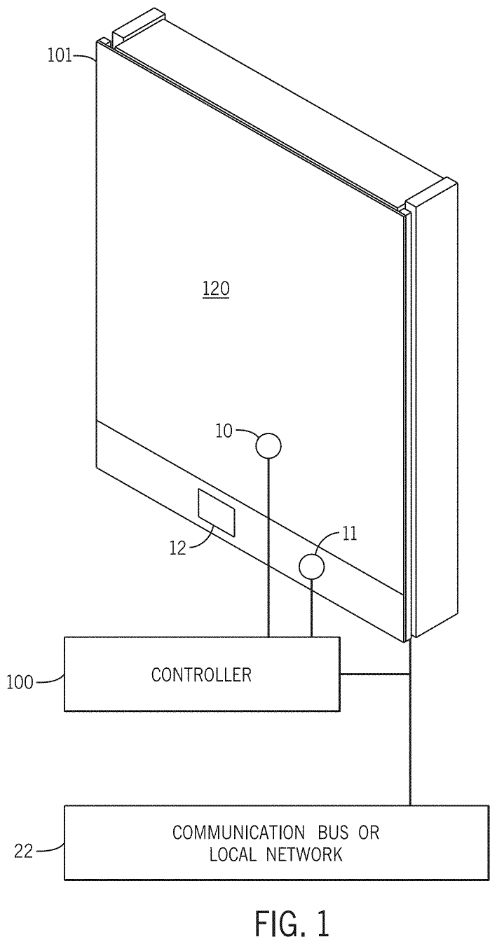

FIG. 1 illustrates an example mirror 101, including a proximity sensor 10, a health sensor 11, a display 12, connected to a controller 100, which may be in communication with a communication network 22. The mirror 101 may include a mirror frame configured to support a mirror substrate that provides a reflection of one or more users in proximity of the mirror cabinet. The proximity sensor 10 and/or the health sensor 11 may be located in a sensor cavity coupled to the mirror frame and configured to support a sensor for detecting a health condition of the one or more users in proximity to the mirror cabinet.

The mirror 101 may include a reflective substrate 120 and a retractable portion 102. The retractable portion 102 may be retractable to be hidden and retracted behind the reflective substrate 120 or otherwise within the mirror 101. The retractable portion 102 may alternatively be a cover that shields the health sensor 11 and/or display 12, which is not illustrated in FIG. 1 but discussed in other embodiments. Additional, different, or fewer components may be included.

Data is collected by the health care mirror 101 by sensor 11. The data may describe the physical characteristics or health characteristics of the user. In some instances, the user requests that a particular type of data be collected. In another instance, the data is collected automatically when the user is in place in front of, in the line of sight of, or in proximity to the health care mirror 101, as detected by the proximity sensor 10. The term “in proximity” may refer to any position within range of the proximity sensor 10. The term “in line of sight” may mean near the health care mirror 101 without any physical obstacle (e.g., a wall) therebetween. The term “in front of” may refer to a position within a predetermined range or area.

The proximity sensor 10 may be a relative distance collection device such as a light or laser scanner. The laser scanner may emit one or more laser pulses that reflect off of objects and are received by the laser scanner. The time of flight for the laser pulses indicates the distance to the objects. For example, the controller 100 may start a timer when the laser is emitted and stop the timer when the laser is received. The round trip time of the laser may be looked up in a table to reference the distance to the object that reflected the laser pulse. Multiple pulses may be emitted and detected. The average round trip time may be calculated by the controller and referenced in the lookup table.

The proximity sensor may detect a presence of an object at a predetermined distance or within a predetermined distance range. That is, the controller 100 may compare the distance of the object accessed from the lookup table with the predetermined distance range. When the distance of the object falls within the predetermined distance range, the controller 100 that an object is in proximity to the bathroom appliance. The proximity sensor may include a microwave or radar sensor. Example predetermined distances may be 28 inches, 3 feet or another distance. The range of the proximity sensor may be cone shaped.

The sensor 11 may be any type of sensor that directly or indirectly detects a characteristic of the health of the user in proximity of the health care mirror 101. While various other sensors are described, in one embodiment the sensor 11 is a camera. The camera may include a lens such as a digital aperture collection device (e.g., camera) or an image collection device with a charge coupled device (CCD) such as an integrated circuit formed on a silicon surface forming light sensitive elements. The image collection device may collect images for facial recognition of the user.

The controller 100 may identify specific regions in images collected by the camera 11. For example, the controller 100 may identify a cheek or upper portion of the check using facial recognition or feature detection/extraction image processing algorithms. In one example, the controller 100 may compare image templates that correspond to human faces or human checks to the specific regions in images collected by the camera 11. The controller 100 may use a sliding window technique where the template (e.g., having a predetermined length and width measured in pixels) is incrementally slid across the detected image (e.g., pixel by pixel or a multiple thereof).

From the image of the controller 100 is configured to calculate characteristics of the health of the user including heart rate, stress level, blood pressure, breathing rates, heart rate variability, oxygen levels, and temperature.

The image collection device may collect images for recognizing an image signature of the user such as the color or shape (e.g., bone density, outline, height, and/or weight) of the user. Other body properties may be determined from the image of the user including skin qualities at a cellular level, signs of hormone imbalance, aging, sun damage, pigmentation, color, inflammation, environmental impacts, or other abnormalities. In another example, the images of the user's muscles are analyzed to determine muscle conditions (e.g., strains, pulls, or tears). The sensor 11 may be a retina scanner configured to scan the eyes of the user. The retina scan may indicate an eye signature for identification of the user. The retina scan may detect characteristics of health such as the blood sugar level of the user.

The controller 100 may compare values in the sensor data to one or more thresholds or ranges in order to identify health characteristics of the user. The controller 100 may send the result of the analysis to the display 12 or user interface, as discussed in more detail below. The display is configured to display the health condition of the user, a state of a timer for the user, and or a message indicating that the sensor is currently collecting data.

The controller 100 may generate a log or journal for the sensor data. That is, the sensor data may be stored in memory with associated timestamps that record when the sensor data was collected. Likewise, the sensor data may be stored with identity of the user, which may be determined using any of the various techniques described herein.

The controller 100 may access the log to determine whether an alert should be generated. The alert may be displayed at the health care mirror 101. The alert may signal to the user that they may be experiencing a health condition. The alert may signal to subsequent users of the health care mirror 101 that the other user is experiencing a health condition. For example, in a home, when one family has a health condition, the other members are alerted through the health care mirror 101. Similarly, in a hotel, dormitory, public restroom, the controller 100 may determine when a user has a health care condition and display an alert for other users to be aware of the risk.

The controller 100 may send the result of the analysis to the communication network 22 directly or through a communication bus to transfer data between the controller 100. The communication network 22 may be coupled to or include a server, a network device (another computer connected to the communication network 22), and a communication bus. Through the communication network 22, the controller 100 may send the message including the result of the analysis or the sensor data to a central controller, which may be implemented by the network device or the server. The central controller may perform the analysis of the sensor data. The central controller may compile sensor data from multiple health care mirrors 101. The central controller may be a cloud device configured to communicate with multiple network devices located in multiple locations (e.g., different homes or businesses) for multiple health care mirrors 101. The central controller may implement a cloud service that coordinates and analyzes data from the multiple health care mirrors 101. The health care mirror 101, or any of the multiple health care mirrors, may receive a report from the central controller that indicates when a health condition is present at any of the other health care mirrors 101. The controller 100 may generate and display an alert at the health care mirror 101 in response to the health condition broadcasted by the central controller. The controller 100 may be configured to analyze data for tracking the user and calculate an instruction for the user in response.

In another embodiment, the health care mirrors may be organized according to geographic region. The controller 100 may identify the position of the health care mirror and include the position with the analysis that is reported to the central controller. The controller 100 may receive the position from a positioning device (e.g., global positioning system (GPS)), the communication network 22 (e.g., IP address), or from user entry. The central controller may organize the data for health conditions according to location. The central controller may identify geographic areas (e.g., neighborhoods, blocks, towns, etc.) that are experiencing statistically significant health conditions. The central controller may use a health condition incident density, which may be measured in incidents per unit area. The central controller may send alerts to the health care mirrors or other mobile devices in the identified geographic areas.

In one example, the analysis of data occurs primarily at the network device, which may be referred to as the local analysis embodiments. In another example, the analysis of data occurs primarily at the server or another remote device, which may be referred to as the remote analysis embodiments. Hybrid embodiments may include a combination of data analysis at the network device and the server.

The sensor data may be aggregated from multiple health care mirrors in order to set the predetermined thresholds for comparison. For example, when the sensor 11 is a thermometer, temperature values may be average to determine the temperature threshold. Different temperature thresholds may be used for different geographic regions. Different temperature thresholds may be used for different demographic groups. That is, a different temperature threshold may be calculate for female users than male users. A different threshold temperature may be calculated for different user age groups.

The server may receive information on the health characteristics of the user from health care mirror 101 along with other data sources such as the health characteristics of other users from other health care mirrors. As described in more detail below, aggregate data from multiple users may be combined to provide assessments of health in larger geographic areas such as neighborhoods, towns, or regions.

The controller 100 may package or pre-process the data in a predetermined format and transmit the data to the server. The network device may filter the data according to type. Example types include audio data, image data, position data, biometric data, ambient data, or other types. For image data, the controller 100 may analyze an image of at least a portion of the user. For position data, the network device may determine a position of the user through analysis of the image (e.g., pattern matching or line detection) or through distance based sensors based on proximity. For biometric data, the network device may collect temperature data (e.g., heat signature) from a temperature sensor or infrared sensor, fingerprint data from a fingerprint sensor, or eye data from a retina scanner. For ambient data, the network device may collect temperature, humidity, or other environmental information.

Any of the sensors of the mirror 101 (e.g., thermometer) may be most accurate when the user is in a particular position or orientation with respect to the mirror 101 and/or the sensor 11. A guide silhouette may be projected or otherwise displayed on the mirror 101. In some examples, the positioning silhouette is etched, painted, or otherwise permanently or semi-permanently applied to the health care mirror 101. The positioning silhouette shows the location that a user should appear in the reflection of the mirror in order to be accurately detected by one or more sensors. In one example, the positioning silhouette is displayed on a liquid crystal display (LCD) that overlays the mirror substrate. Thus, the positioning silhouette may be variable at the controller 100 may control the location of the positioning silhouette.

In some examples, the positioning silhouette is not visible but rather determined dynamically by the controller based on the user identity (e.g., age or size of the user) or based on the sensor data. The controller 100 is configured to identify a selected user of the one or more users from at least one image of the time series of images and access a profile based on the selected user. The controller 100 may detect a position or orientation of the one or more users from at least one image of the time series of images and generate an alignment instruction based on the detected position or orientation.

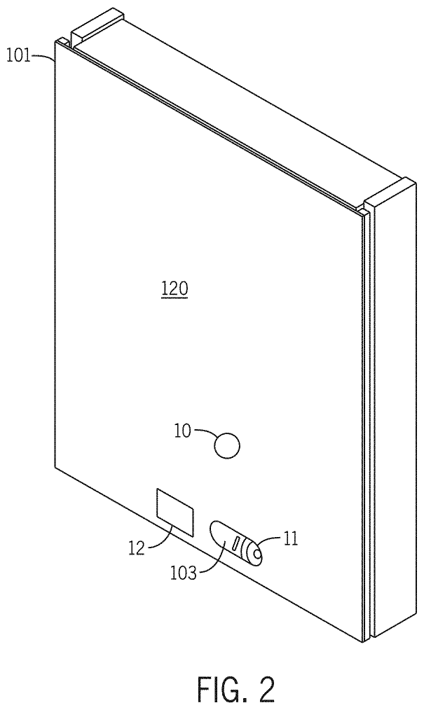

FIG. 2 illustrates another example health sensing mirror 101. In the example of FIG. 2, the retractable cover 102 is omitted. Instead a retractable lens cover 103 is shown. The retractable lens cover 103 may be opened or closed based on data collected by the proximity sensor 10. When the user is initially in proximity to the health sensing mirror 101 the retractable lens cover 103 is opened so that the sensor 11, such as a camera, collects data. After a predetermined amount of time sufficient to collect the sensor data, the retractable lens cover 103 is closed. After processing, the health conditions of the users are provided to display 12.

The lens cover 103 may be opened and closed using a variety of mechanisms. In one example the lens cover 103 is biased open by a spring. A solenoid is coupled to the lens cover 103. When the solenoid is actuated, it slides the lens cover along a grove against the bias force of the spring in order to close the lens cover 103.

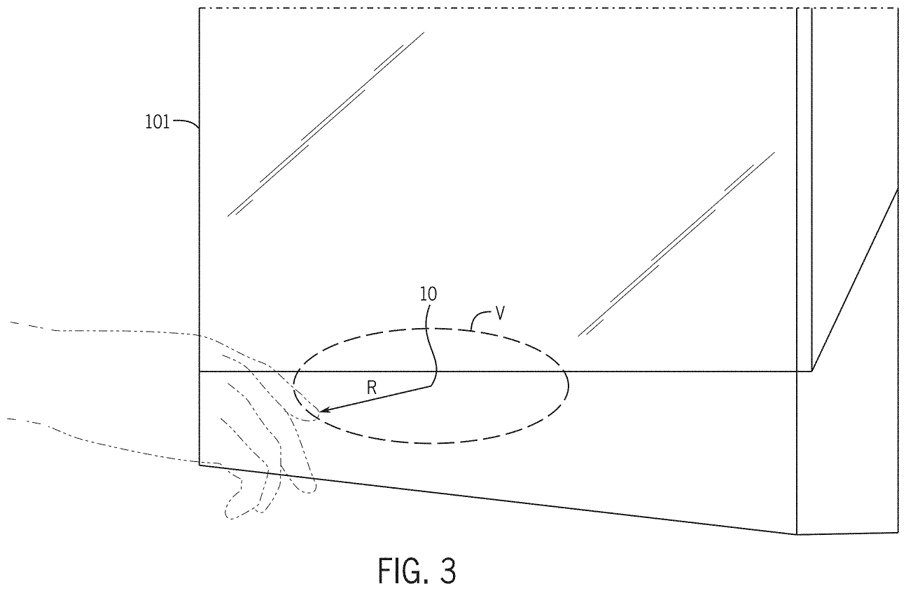

FIGS. 3-6 illustrate another example health sensing mirror. FIG. 3 illustrates a proximity trigger for a retractable sensor assembly of an example health sensing mirror. The proximity may be configured to detect object within a radius R. Thus, the detection region or volume V may have a semi-spherical or semi-ellipsoid shape.

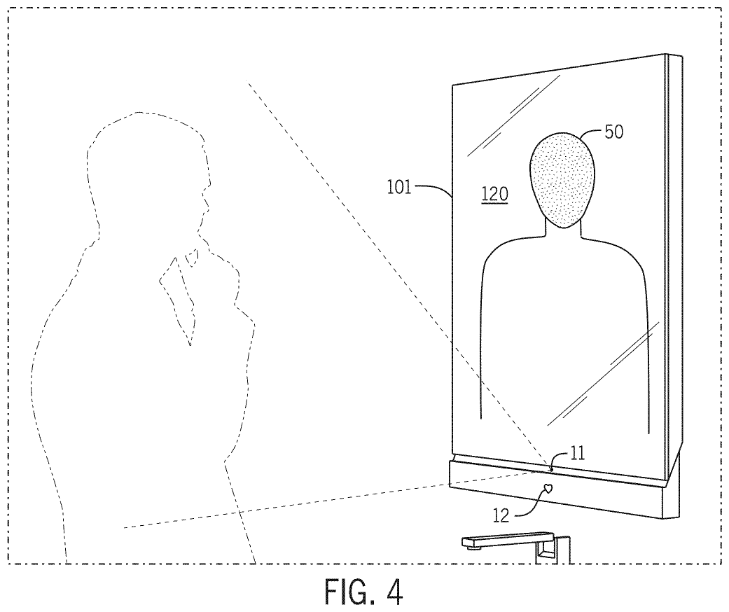

FIG. 4 illustrates a field of view for a sensor 11, such as a camera, of the example health sensing mirror. The field of view may be selectable for privacy reasons. The field of view may be a configurable angle such as 20 degrees to 135 degrees. The angle may be selected and configured by the controller 100. The angle may be selected and configured manually by the user. For example, the user may install or remove a variable lens cap, and/or the user may rotate a variable lens cap that changes the field of view as it is rotated.

FIG. 4 also illustrates a guide silhouette 50 that may be display on the mirror 101. There may be a semitransparent display overlayed over parts or all of the substrate 120. The guide silhouette 50 may also be painted or otherwise affixed to the substrate 120. The guide silhouette 50 gives the user a target to position his or her reflection in the mirror 101 to optimize the collection of data.

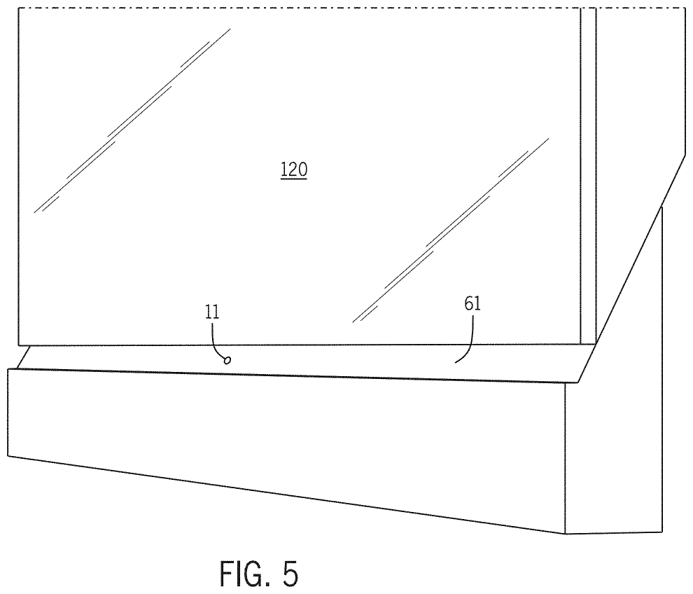

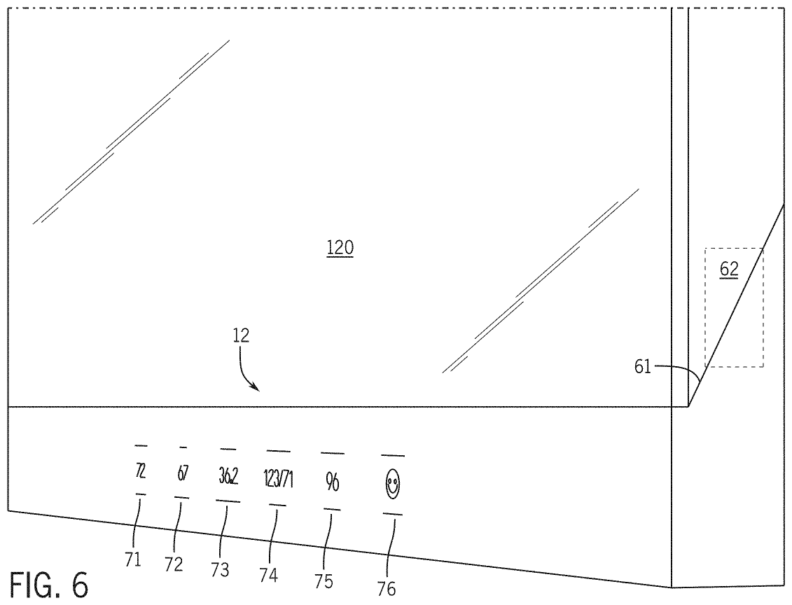

FIG. 5 illustrates an opened retractable sensor assembly 61. FIG. 6 illustrates a closed retractable sensor assembly 61. The retractable sensor assembly 61 may be a movable component that moves with respect to the health sensing mirror 101 to be exposed or hidden behind another component such as the substrate 120. The movable component may be moved forward to cover the one or more sensors of the health sensing mirror 101 as shown by FIG. 6. The movable component may be retracted to reveal the one or more sensors of the health sensing mirror 101 as shown by FIG. 6. Alternatively, a plate or cover may be the movable component that is moved to expose or hide the sensors such as the sensor 11.

A drive mechanism 62 is configured to cover or reveal the sensor 11. The drive mechanism 62 may include one or more solenoids or one or more motors such as a stepper motor. The drive mechanism 62 lowers or raises the sensor cavity with respect to the mirror frame. The retractable sensor assembly 61 may slide and/or pivot along a track and an axis to move between the cover position in FIG. 6 and the retracted position in FIG. 6. In some examples, such as the embodiment of FIG. 2, the drive mechanism 62 retracts a lens cover for the sensor. In some examples, such as the embodiment of FIGS. 3-6, wherein the drive mechanism 62 lowers a sensor cavity cover. In other examples, the sensor cavity may be raised or lowered by the drive mechanism 62.

Data from the proximity sensor 10 and/or the controller 100 triggers the drive mechanism 62 to reveal the sensor 11. That is, the controller 100 may send a command to the drive mechanism 62 in response to data received from the proximity sensor 10.

The drive mechanism 62 may be powered by a battery, plugged into an electric outlet, or may be hardwired into the wiring of a building.

The health sensing mirror 101 may include a timer for the transition from the covered state of FIG. 5 to the retracted state of FIG. 6. The timer may be implemented by controller 100. The timer may be integrated with a drive circuit of the drive mechanism 62.

In one example, the duration of the timer is a set value. For the example, the timer starts elapsing with the proximity sensor 10 and/or the controller 100 triggering the drive mechanism 62 to reveal the camera or other sensor 11. Alternatively, the movement of the drive mechanism 62 may initiate the timer to start running. When the set value for the timer, elapses the time sends a signal to the controller 100 or the drive mechanism 62 that causes the sensory cavity to be concealed, the sensor cavity to close, or a cover to be moved in front of the sensor cavity.

The duration of the time may be variable. The controller 100 may determine during the processing of the sensor data, when enough data has been collected to determine the one or more characteristics of the user. When enough data has been collected, the controller 100 sends a command to the drive mechanism that causes the sensory cavity to be concealed, the sensor cavity to close, or a cover to be moved in front of the sensor cavity. The timer duration may be selectable by the user. In other examples, the controller 100 may define the time through a user input. Alternatively, the controller 100 may define the time through an iterative analysis. Future times for the timer may be defined according to past times that were required. The past times may be specific to the users that have been analyzed by the health sensing mirror 101. The timer duration may be set according to the quantity and type of image processing algorithms that are applied to the images collected by the sensor 11.

FIG. 6 also illustrates the display 12, which may include a screen, a series of light emitting diodes (LEDs), or another display. The display 12 may include multiple portions and each portion may be assigned to a different health characteristic. The display 12 may include multiple health characteristics such as a heart rate (e.g., beats per minute) 71, a heart rate variability 72 (e.g. milliseconds), a body temperature 73 (e.g., degrees Celsius or Fahrenheit), a blood pressure 74, an oxygen saturation 75 (e.g., percentage) and/or a stress level 76 (e.g., relaxed, moderately stressed, highly stressed). Other health characteristics such as respiration or blood tissue volume may be used. As shown in FIG. 6, a different icon, textual message, and/or data may be displayed for each of heart rate, stress level, blood pressure, breathing rate, heart rate variability, oxygen level, and temperature.

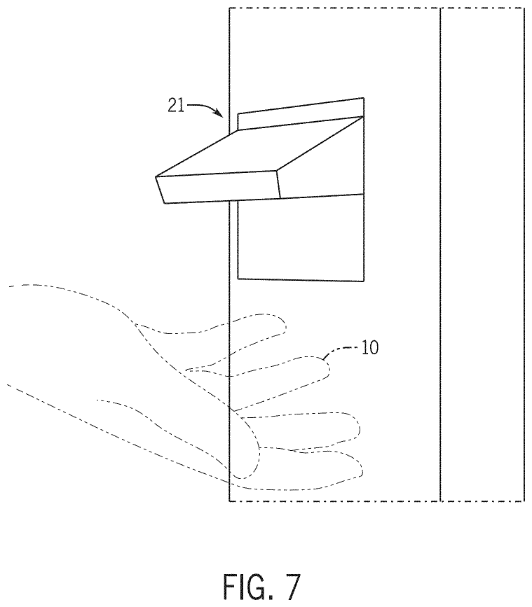

FIG. 7 illustrates an example sanitizer dispenser 21 and a proximity sensor 10, which may be incorporated into the health care mirror 101. The dispenser 21 may include a nozzle that dispenses hand sanitizer. The nozzle may dispense sanitizer in response to the proximity sensor 10. That is, when a user (e.g., hand) is detected by the proximity sensor 10 in a predetermined distance range (e.g., 5 inches or 12 centimeters) from the health care mirror 101, the sanitizer dispenser 21 dispenses sanitizer. The dispenser 21 may be operated by controller 100 that processes the data from the proximity sensor 10 and performs one or more calculations or comparisons to determine whether the user is present in range.

In one example, the dispenser 21 may include a drive mechanism configured to cause the dispenser 21 to fold out from the cabinet of the mirror 101 in order to dispense sanitizer. The drive mechanism also retracts the dispenser 21 when the sanitizer is not being dispensed. The drive mechanism may include a solenoid, a motor, and/or a drive train that is actuated by a command from the controller 100.

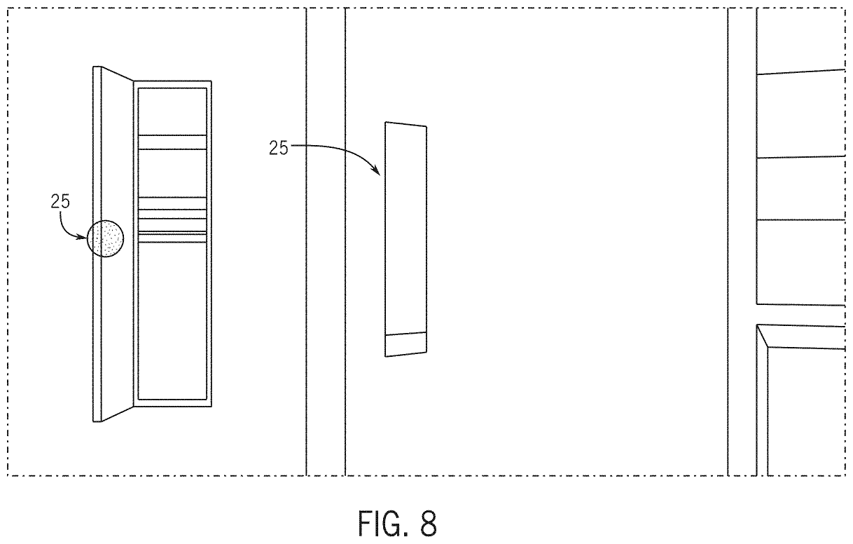

FIG. 8 illustrates a cartridge 25 for the sanitizer dispenser 21. The cartridge 25 includes a container and a nozzle. The nozzle may be the same nozzle that dispenses the sanitizer. The cartridge may be replaceable (e.g., when the container is empty). The cartridge may include an electrical contact for communication with the controller 100 and/or the proximity sensor 10.

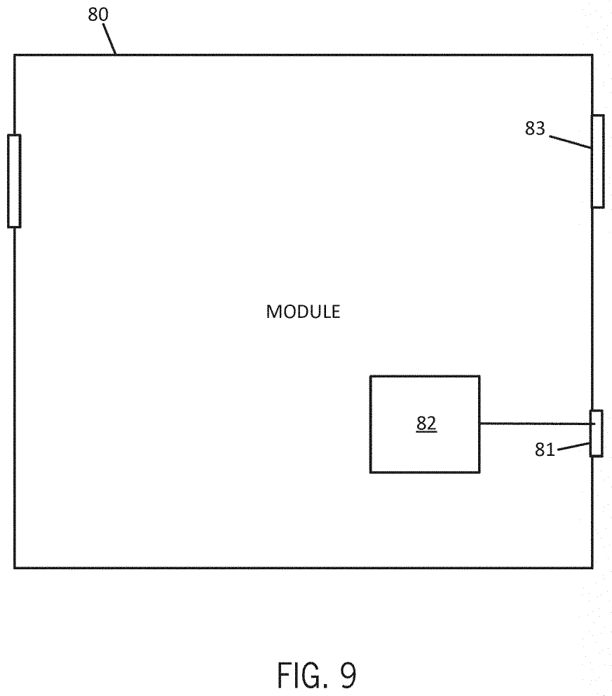

FIGS. 9-11 illustrate example modules for a mirror. FIG. 9 illustrates a module 80. A variety of different types of modules that perform different functions may be used. The module 80 may include any one or a combination of shelving, drawers, hangers, enclosures, sanitation, ultraviolet illumination, visible light illumination, near visible light illuminating, wireless charging, wired charging, disinfectant, heating, cooling, or other functions. The module 80 may be configured to fit inside the mirror 101. The module 80 may be placed in the mirror 101 in a variety of different positions. The user may prefer to mount certain modules for certain functions at a high level and mount other modules for other functions at a low level.

The module 80 may include an electrical contact 81 in a predetermined position. The predetermined position is aligned to make contact with a power supply of the mirror 101. In one example, the electrical contact 81 is an electrical conductor that comes in contact with an electrical conductor of the mirror 101. In another example, the electrical contact 81 is an inductive element that receives a magnetic field generated by the mirror 101. There may be multiple electrical contacts on the module 80 so that the module can be installed in different orientations (e.g., reversible horizontally and/or vertically). The power transferred from the mirror 101 to the module 80 may be DC or AC.

The module 80 may include coupling mechanism 83. The coupling mechanism 83 may include a support such as a rail or slide. Through the coupling mechanism 83, the module 80 is mountable and dismountable from the mirror 101. Through either a switch connected to the coupling mechanism 83 or contact of the electrical contact 81, the controller 100 may detect when a module is mounted or removed from the mirror. In addition, the controller 100 may detect what type of module has been connected (e.g., RFID, bar code, or other signature).

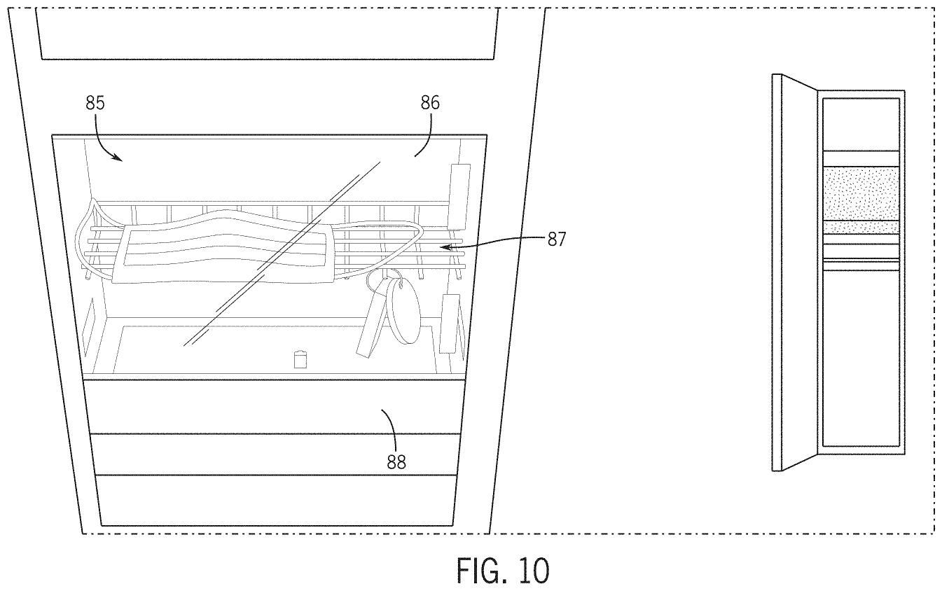

FIG. 10 illustrates an example module 85 including a light 86, a rack 87, and a charging platform 88. The light 86 may be an ultraviolet light or other sanitizing light. The rack 87 may be a metal rack or antibacterial surface. The rack 87 may be treated to prevent adherence of certain materials such as viruses. The rack 87 may used to store certain items such as face mask, phone, keys, etc. that are placed on the rack 87 when returning to the home. The charging platform may include electrical ports (e.g., universal serial bus) or inductive chargers for charging a mobile device (e.g., cellphone). The charging of the mobile device by the charging platform 88 and the sanitizing of the mobile device by the light 86 may occur at the same time.

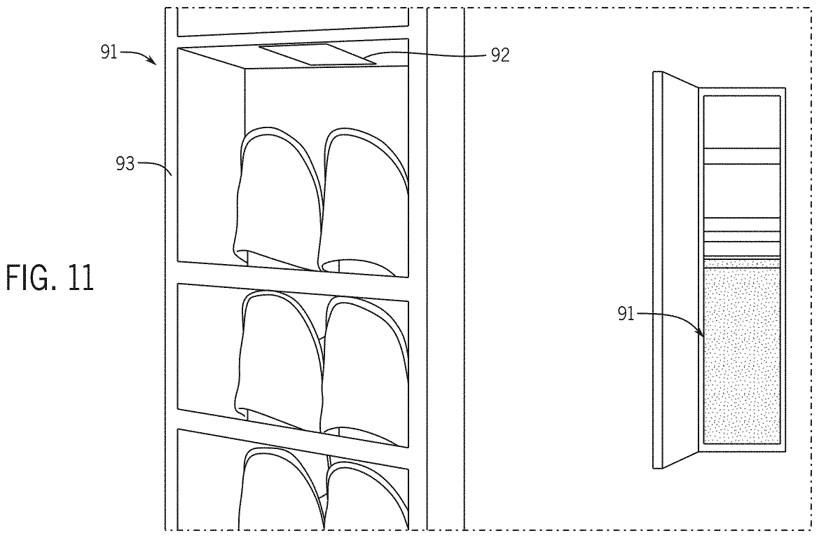

FIG. 11 illustrates an example module 91, including a light 92 and a hanger 93. The light 92 may illuminate the module 91. The light 92 may also dry items that are wet. A separate fan or blower for drying may be included. The hanger 93 may provide a place to store certain items such as shoes, slippers, hats, or other items. The items may be common items used when returning to the home (e.g., slippers) or when exiting the home (e.g., face mask, purse).

Another point for data collection in the bathroom is the toilet. Like the mirror, the daily routine of many people includes at least one brief stop at the toilet. In many cases, a portion of the toilet (e.g., toilet seat) makes direct contact with the user. The toilet may be harnessed as a portal into the lives of users. These visits provide an opportunity to collect data from the users on a regular basis. The data may describe the health of the user. Tracking the health of the user over time provides a wealth of opportunities for technological improvements in a variety of technology areas as described in the following embodiments.

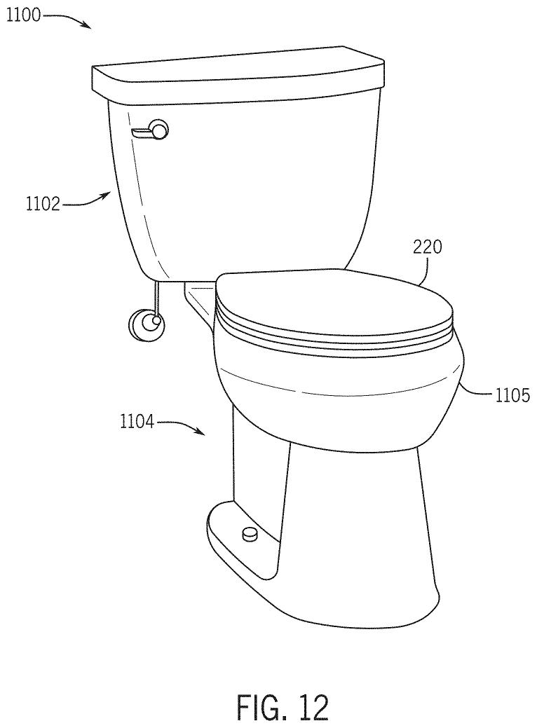

FIG. 12 illustrates a toilet 1100 including a health sensing toilet seat 220. The toilet 1100 may include a tank (e.g., container, reservoir, etc.), shown as a tank 1102, and a pedestal (e.g., base, stand, support, etc.), shown as a pedestal 1104. The tank 1102 may be coupled to, and supported by, the pedestal 1104, which may be positioned on a floor. In some embodiments, the tank 1102 and the pedestal 1104 may be formed together as a single component. The tank 1102 is configured to receive water (e.g., via a fill valve of the toilet 1100, etc.) and store the water in between flushes. The pedestal 1104 includes a bowl 1105 and may be configured to receive the water from the tank 1102 to flush contents of bowl into a sewage line. In some embodiments, the pedestal 1104 may be mounted on the wall of a lavatory and the bowl 1105 may be configured to receive water from a fluid supply source such as a household water supply.

The bowl 1105 of the pedestal 1104 includes a sump (e.g. a receptacle) and an outlet opening, wherein water and waste is collected in the sump until being removed through the outlet opening, such as when the contents of the bowl 1105 are flushed into a sewage line. The toilet 1100 further includes a trapway, the trapway being fluidly connected to the bowl 1105 via the sump. The trapway fluidly connects the sump to the outlet opening and may form a siphonic seal for a flush cycle.

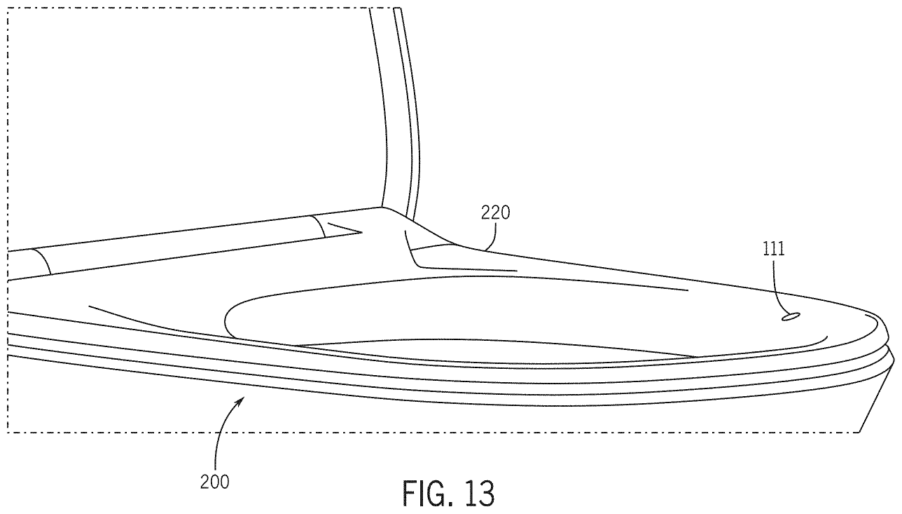

FIG. 13 illustrates an example health sensing toilet seat 220 having a sensor 111. The toilet seat 220 may be coupled to toilet 200. The sensor 111 may be placed a sensor cavity within the toilet seat frame. The toilet seat frame is configured to support a user and may have solid construction throughout except the sensor cavity and one or more additional cavities such as a battery cavity and/or a controller cavity. Alternatively or in addition to the latter, the sensing toilet seat 220 may be connect an electrical outlet by a cord. Any of the examples above, regarding the analysis of sensor data, display of health conditions, log of health conditions, and local or regional logs or alerts, or other features, described above with regarding to the health sensing mirror 101, may be applied to the health sensing toilet seat 220.

A controller 100 is configured to analyze data received from the sensor. The controller 100 may be mounted inside the toilet seat frame or externally. The controller 100 may be found at an external device such as a mobile device or server.

The sensor 111 may be a photoplethysmography (PPG) sensor or another type of optical sensor. The PPG sensor may detect various properties that are used individually or in combination for determining heart rate, stress level, blood pressure, breathing rate, heart rate variability, oxygen level, and temperature. In one technique, the PPG sensor is configured to detect blood volume changes in the microvascular bed of tissue. In one technique, the PPG sensor may illuminate the tissue or skin of the user and measure the absorption of light by the tissue. The change in volume caused by the pressure pulse of a heartbeat is detected by illuminating the user with a light-emitting diode (LED). The amount of deflect may be measured by a photodiode. Blood flow to the skin is affected by other physiological systems, and so these measurements may determine a variety of health characteristics.

The sensor 111 may be triggered to start collecting data in response to a pressure sensor that is also embedded in the seat. The pressure sensor may be a mechanical sensor that makes an electrical connection. The electrical connect may connect the sensor 111 to the controller 100.

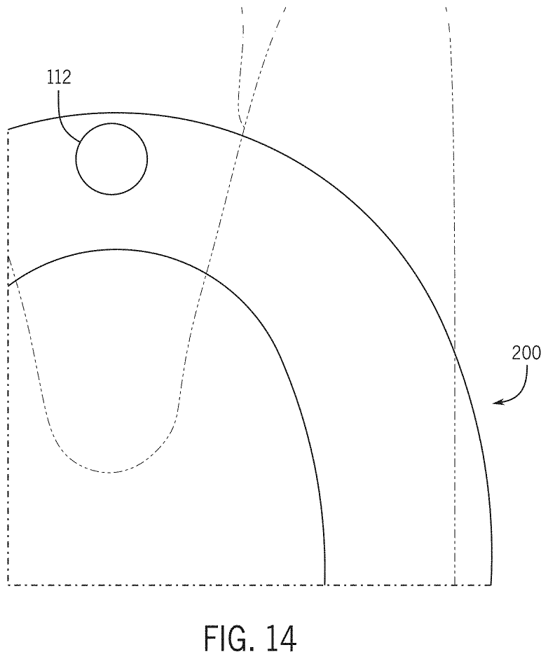

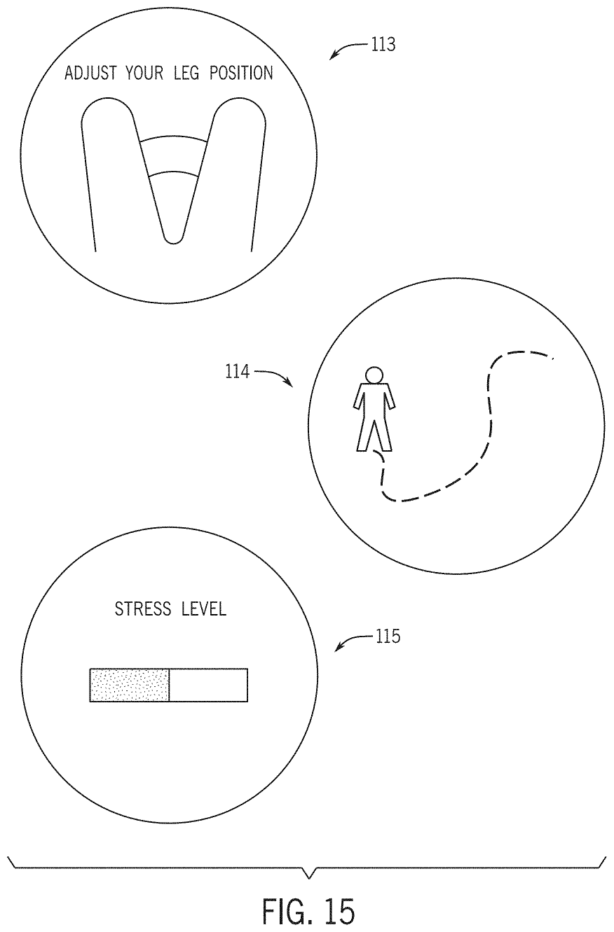

The toilet seat 220 may include a display 112 on a surface of the toilet seat frame. FIG. 14 illustrates an example display for a health sensing toilet seat. The display 112 may include a liquid crystal display (LCD) or a series of LCDs. FIG. 15 illustrates example images for the display for the health sensing toilet seat. The images may include an instructional image 113 that instructs the user how to better position the user's legs or thighs in order to make contact with the sensor 111 properly. The instructional image 113 may present an image of legs on the toilet seat for the user to mimic. The instructional image 113 may include an arrow to instruct the user how to move. The instructional image 113 may include a red light when more data (better positioning) is needed and a green letter when sufficient data (recommend positioning) has been achieved.

The images may include a distraction image 114 or animation. The animation may be a short video or sequence of lights that distracts the user for enough time to collect the data. The period of time of the distraction image 114 or animation may be similar to the timer described herein.

The images may include a health condition indicator 115. The health condition indicator may describe a value for heart rate, stress level, blood pressure, breathing rate, heart rate variability, oxygen level, and temperature.

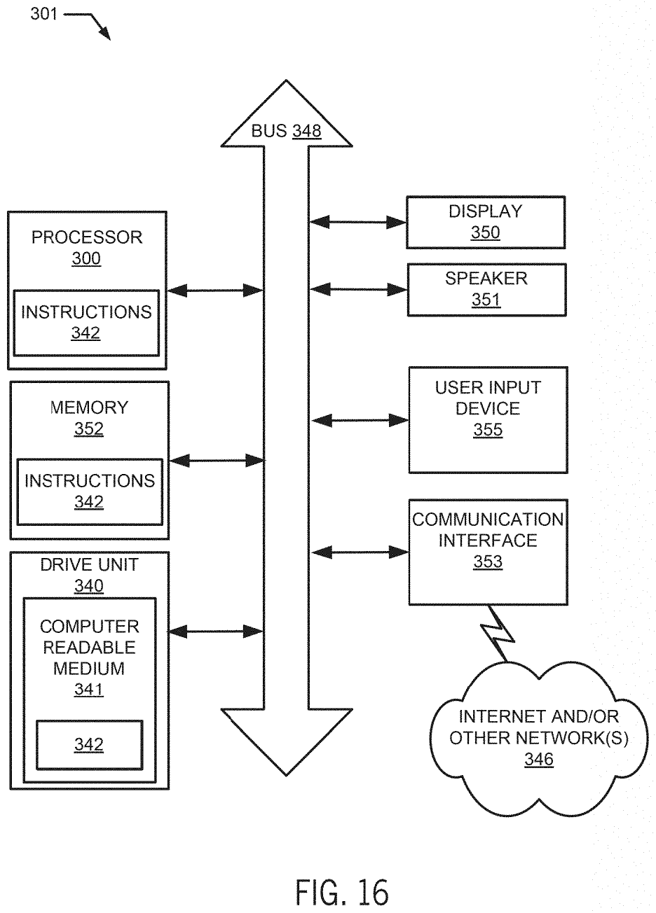

FIG. 16 illustrates an example embodiment for a control system 301 (e.g., controller 100) for the health sensing mirror or toilet seat. The control system 301 may include a processor 300, a memory 352, and a communication interface 353 for interfacing with devices or to the internet and/or other networks 346. In addition to the communication interface 353, a sensor interface may be configured to receive data from the sensor 11 or data from any source for tracking a user in a bathroom in proximity to the health care mirror 101.

The components of the control system 301 may communicate using bus 348. The control system 301 may be connected to a workstation or another external device (e.g., control panel) and/or a database for receiving user inputs, system characteristics, and any of the values described herein. Optionally, the control system 301 may include an input device 355 and/or a sensing circuit in communication with any of the sensors. The sensing circuit receives sensor measurements from as described above. The input device 355 may include a touchscreen coupled to or integrated with the mirror, a keyboard, a microphone for voice inputs, a camera for gesture inputs, and/or a holographic interface coupled to or integrated with the mirror.

Optionally, the control system 301 may include a drive unit 340 for receiving and reading non-transitory computer media 341 having instructions 342. Additional, different, or fewer components may be included. The processor 300 is configured to perform instructions 342 stored in memory 352 for executing the algorithms described herein. A display 350 may be supported by the mirror frame. The display 350 may be combined with the user input device 355.

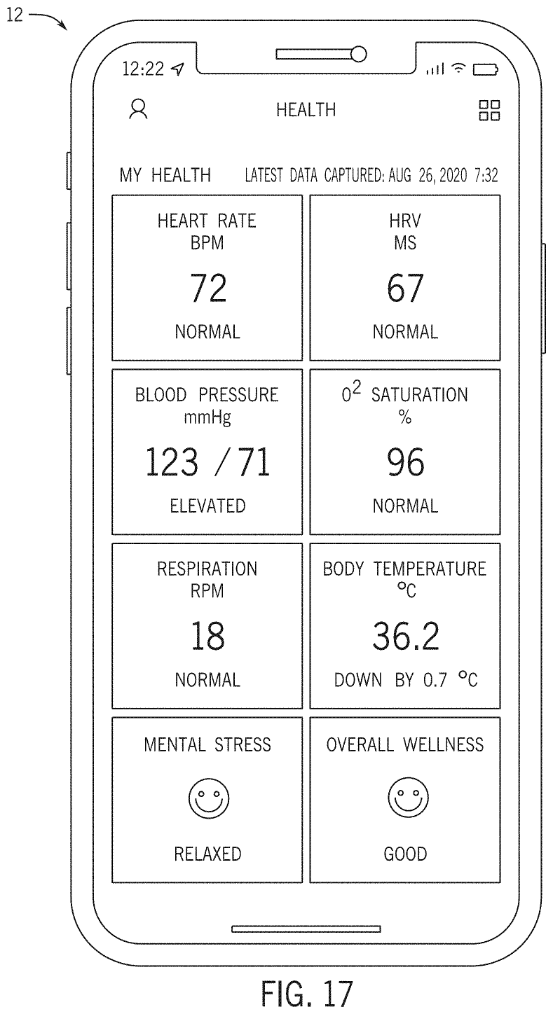

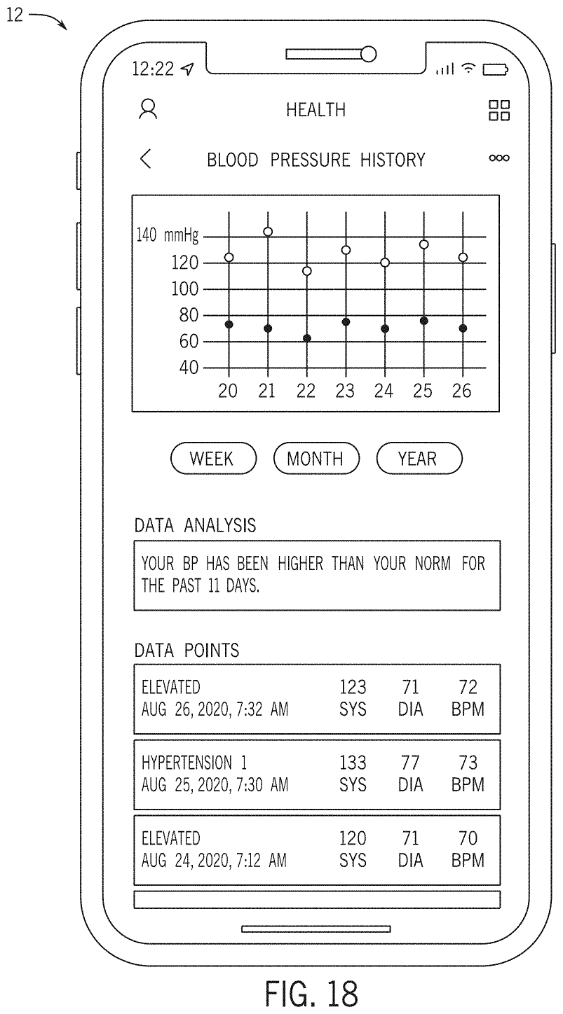

FIGS. 17 and 18 illustrate a mobile device as the display 12. The mobile device may provide various combinations of the health condition data to the user. The mobile device may provide log information, alerts, or other information described herein. Through the mobile device, the user may provide inputs to the mirror and/or toilet seat. One input may set the timer for collecting data. One input may impact what is displayed at the local display of the mirror 101 or toilet seat 122. The input may select the instructional images or health conditions that are displayed.

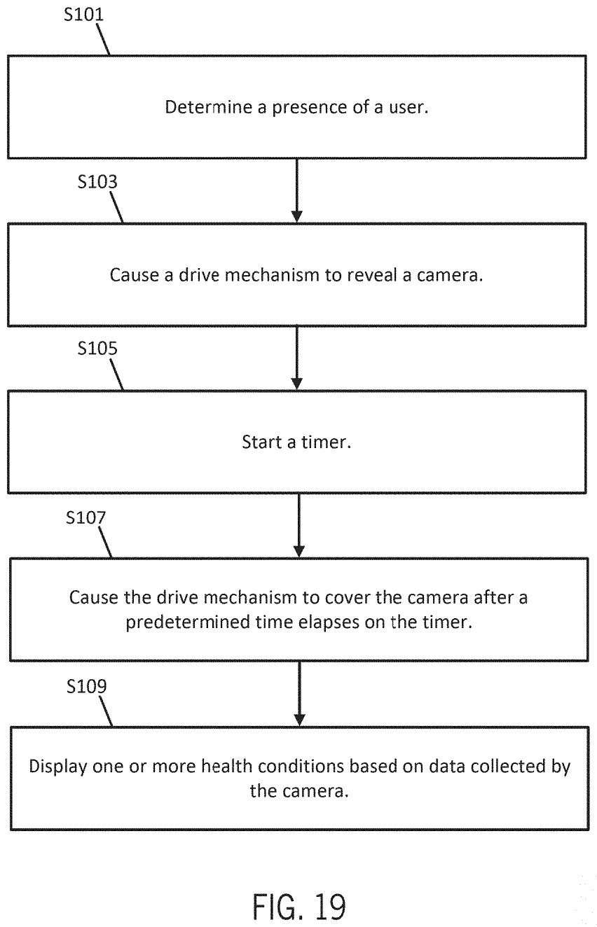

FIG. 19 illustrates an example flow chart a temperature tracking algorithm for the health sensing mirror 101, the health sensing toilet seat 220, or another bathroom sensing appliance. Other characteristics of the user besides temperature may be detected. Examples include heart rate, stress level, blood pressure, breathing rate, heart rate variability, oxygen level, and temperature. The acts of the flow chart may be performed by any combination of the controller 100, the network device or the server. Portions of one or more acts may be performed by the appliance. Additional, different, or fewer acts may be included.

At act S101, the controller 100 (e.g., through processor 300) determines the presence of a user, for example, based on data received from the proximity sensor. The controller 100 is configured to analyze sensor data from the proximity sensor to determine whether the user is near the bathroom appliance. The proximity may detect movement within a predetermined area or radius from the bathroom appliance. The controller 100 may initiated a data collection process in response to the detection of the user with the proximity of the bathroom appliance.

Alternatively, the bathroom appliance may start the data collection process according to a predetermined time such as a time of day, day of the week, or a time interval. In addition or in the alternative, the data collection process may be started in response to a user input. For example, the user may be prompted with a message on the display to authorize the collection of data. The controller 100 is configured to identify a user input that authorizes the data collection.

The collection of data may include multiple acts. For example, at act S103, the controller 100 (e.g., through processor 300) instructs a drive mechanism to reveal a camera. In addition or in the alternative to the camera, sensors may be included. Certain sensors may be used in the health sensing mirror 101 and other types of sensors used in the health sensing toilet seat 220. A PPG sensor may detect various properties that are used individually or in combination for determining heart rate, stress level, blood pressure, breathing rate, heart rate variability, oxygen level, and temperature. In one technique, the PPG sensor is configured to detect blood volume changes in the microvascular bed of tissue. In one technique, the PPG sensor may illuminate the tissue or skin of the user and measure the absorption of light by the tissue. The change in volume caused by the pressure pulse of a heartbeat is detected by illuminating the user with a light-emitting diode (LED). The amount of deflect may be measured by a photodiode.

In other examples, other sensors may be revealed, opened, or activated. Activating a sensor may include providing electrical power to the sensor or sampling the data outputted by the sensor. Thus, in response to the data collection process, the controller 100 may begin to sample data from the sensor.

In another intermediate act, after the sensor (e.g., camera) has started collecting data but before the analysis of the data has begun, the controller 100 may determine whether the user is in alignment with the sensor in a manner sufficient to collect reliable data. For example, a guide silhouette for alignment of the user with the mirror cabinet may be illuminated or otherwise displayed. The controller 100 is configured to analyze sensor data collected by the camera in response to alignment of the user with the mirror cabinet.

Data collected by the camera may be analyzed by the controller 100 or another device. One or more health conditions are determined by the analysis.

At act S105, the controller 100 (e.g., through processor 300) starts a timer. Alternatively, the camera may measure one or more biometrics of the user, which requires a predetermined amount of time. The camera collects at least two images of the user's face in order to calculate changes in skin color and movement, which requires the predetermined amount of time.

At act S107, the controller 100 (e.g., through processor 300) after a preset time elapses, instruct the drive mechanism to cover the camera. The drive mechanism may include a motor, solenoid, gear train or rother device that move a cover plate for either the camera lens or for the sensor array.

At act S109, the controller 100 (e.g., through processor 300) causes the display to display health characteristics based on data from the camera. A first characteristic may be displayed using a first icon and a second health characteristic may be displayed using a second icon. A first characteristic may be displayed using a first alphanumeric value and a second health characteristic may be displayed using a second alphanumeric value. The health characteristics may include heart rates, breath rates, tissue state, mood, or other indicators of the health of the user.

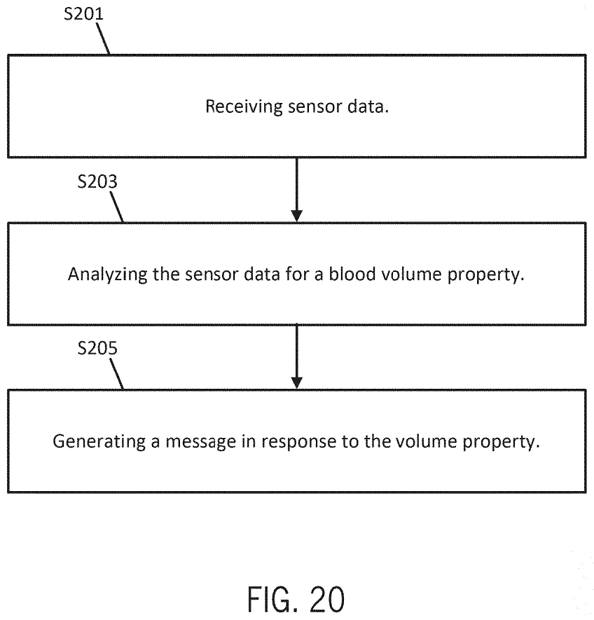

FIG. 20 illustrates another flow chart for the control system 301 (e.g., controller 100) for the health sensing mirror or toilet seat. A sensor interface may be configured to receive data from the sensor 11 or data from any source for tracking a user in a bathroom in proximity to the health care mirror 101.

At act S201, the controller 100 receives sensor data from a bathroom device. The sensor data may be generated at a camera, a PPG sensor, an infrared sensor, or any device configured to detect a property of the tissue of the user. One example property is the blood volume property, which describes a quantity of blood in the tissue. The infrared sensor may be configured to measure a hemoglobin concentration and/or a normalized tissue hemoglobin index.

At act S203, the controller 100 analyzes the sensor data for a blood volume property of a user. The controller 100 may compare the blood volume property to one or more thresholds or ranges. For example, a first range may represent a first health condition and a second range may indicate a second health condition. For example, the first range may represent high blood pressure and the second range may represent normal blood pressure. In another example, the first range may indicate normal oxygen levels and the second range may indicate low oxygen levels. The controller 100 may monitor the blood volume property over time. When the blood volume property changes more than a specific amount over a time period, the controller 100 may identify that there has been a change in a microvascular bed of tissue of the user, which may be associated with one or more health conditions.

At act S205, the controller 100 generates a message in response to the blood volume property of the user. The message may cause the display of an alert to the user. The message may be sent to an external device that is associated with multiple users. For example, the external device may be a central computer or server associated for an organization, municipality, or geographic area.

In one example, the external device includes a hospital computer that is connected to a plurality of bathroom devices in various patient rooms. As the patients stops to look in the mirror, or sits on a toilet seat, data is collected and aggregated at the hospital computer. The hospital computer may alert a medical professional, dispense medicine, or amend a patient record in response to the collected data. A similar system may be used in a hotel, dormitory, barracks, or apartment building.

In one example, the external device may be a government computer that is connected to a plurality of bathroom devices in various homes. The government computer may collect data from citizens and provide services in response. For example, the government may assign vaccine distribution, emergency medical staff, or temporary hospital facilities in response to the collected data.

Processor 300 may be a general purpose or specific purpose processor, an application specific integrated circuit (ASIC), one or more programmable logic controllers (PLCs), one or more field programmable gate arrays (FPGAs), a group of processing components, or other suitable processing components. Processor 300 is configured to execute computer code or instructions stored in memory 352 or received from other computer readable media (e.g., embedded flash memory, local hard disk storage, local ROM, network storage, a remote server, etc.). The processor 300 may be a single device or combinations of devices, such as associated with a network, distributed processing, or cloud computing.

Memory 352 may include one or more devices (e.g., memory units, memory devices, storage devices, etc.) for storing data and/or computer code for completing and/or facilitating the various processes described in the present disclosure. Memory 352 may include random access memory (RAM), read-only memory (ROM), hard drive storage, temporary storage, non-volatile memory, flash memory, optical memory, or any other suitable memory for storing software objects and/or computer instructions. Memory 352 may include database components, object code components, script components, or any other type of information structure for supporting the various activities and information structures described in the present disclosure. Memory 352 may be communicably connected to processor 300 via a processing circuit and may include computer code for executing (e.g., by processor 300) one or more processes described herein. For example, memory 298 may include graphics, web pages, HTML files, XML files, script code, shower configuration files, or other resources for use in generating graphical user interfaces for display and/or for use in interpreting user interface inputs to make command, control, or communication decisions.

In addition to ingress ports and egress ports, the communication interface 353 may include any operable connection. An operable connection may be one in which signals, physical communications, and/or logical communications may be sent and/or received. An operable connection may include a physical interface, an electrical interface, and/or a data interface. The communication interface 353 may be connected to a network. The network may include wired networks (e.g., Ethernet), wireless networks, or combinations thereof. The wireless network may be a cellular telephone network, an 802.11, 802.16, 802.20, or WiMax network, a Bluetooth pairing of devices, or a Bluetooth mesh network. Further, the network may be a public network, such as the Internet, a private network, such as an intranet, or combinations thereof, and may utilize a variety of networking protocols now available or later developed including, but not limited to TCP/IP based networking protocols.

While the computer-readable medium (e.g., memory 352) is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored. The computer-readable medium may be non-transitory, which includes all tangible computer-readable media.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

1. A mirror cabinet comprising:
a mirror frame configured to support a mirror substrate that provides a reflection of one or more users in proximity of the mirror cabinet;
a sensor cavity coupled to the mirror frame and configured to support a sensor for detecting a health condition of the one or more users in proximity to the mirror cabinet; and
a controller configured to analyze data received from the sensor to determine the health condition.

2. The mirror cabinet of claim 1, wherein the sensor is a camera and the controller performs an image processing algorithm on images collected by the camera in order to determine the health condition of the one or more users.

3. The mirror cabinet of claim 1, further comprising:
a drive mechanism configured to cover or reveal the sensor.

4. The mirror cabinet of claim 3, wherein the drive mechanism lowers or raises the sensor cavity with respect to the mirror frame.

5. The mirror cabinet of claim 3, wherein the drive mechanism retracts a lens cover for the sensor.

6. The mirror cabinet of claim 3, wherein the drive mechanism lowers a sensor cavity cover.

7. The mirror cabinet of claim 3, further comprising:
a proximity sensor that triggers the drive mechanism.

8. The mirror cabinet of claim 1, further comprising:
a display configured to display the health condition.

9. The mirror cabinet of claim 8, wherein the display also provides a state of a timer for the user.

10. The mirror cabinet of claim 8, wherein the display also provides a message indicating that the sensor is currently collecting data.

11. A method for a mirror cabinet, the method comprising:
determining a presence of a user;
instructing a drive mechanism to reveal a camera for collection of user data;
starting a timer; and
in response to a preset time elapsing at the time, instructing the drive mechanism to cover the camera.

12. The method of claim 11, further comprising:
displaying health characteristics based on an analysis of data from the camera.

13. The method of claim 11, wherein determining the presence of the user comprises:
analyzing sensor data from a proximity sensor.

14. The method of claim 11, wherein determining the presence of the user comprises:
identifying a user input for data collection.

15. The method of claim 11, further comprising:
displaying a guide silhouette for alignment of the user with the mirror cabinet.

16. The method of claim 15, further comprising:
analyzing user data collected by the camera in response to alignment of the user with the mirror cabinet.

17. A toilet seat comprising:
a toilet seat frame configured to support a user;
a sensor cavity within the toilet seat frame and configured to support a sensor for detecting a health condition of the user in proximity to the toilet seat; and
a controller configured to analyze data received from the sensor.

18. The toilet seat of claim 17, further comprising:
a display on a surface of the toilet seat frame.

19. The toilet seat of claim 17, wherein the sensor is configured to measure a blood volume property of a user.

20. The toilet seat of claim 17, wherein the health condition includes heart rate, blood pressure, breathing rate, heart rate variability, or oxygen level.

21-32. (canceled)
